(19) United States
(12) Patent Application Publication
(10) Pub. No.: US 2009/0277450 A1
(43) Pub. Date: Nov. 12, 2009

(54) Nontoxic antimicrobial filters containing triclosan

(75) Inventors: Pierre J. Messier, Quebec (CA); David Ohayon, Dollard-des-Ormeaux (CA)

Correspondence Address: GOODWIN PROCTER LLP;ATTN: PATENT ADMINISTRATOR, 620 Eighth Avenue, NEW YORK, NY, 10018, US

(73) Assignee: Triosyn Holding Inc., Williston, VT (US)

(21) Appl. No.: 12/384,432

(22) Filed: Apr. 3, 2009

Related U.S. Application Data
(60) Provisional application No. 61/072,915, filed on Apr. 3, 2008.

(51) Int. Cl. A41D 13/11; A61M 16/00

(52) U.S. Cl. 128/205.27

(57) ABSTRACT

The present invention comprises nontoxic, environmentally friendly triclosan-containing antimicrobial filters composed of two layers, a filtering layer and a backing layer used to trap any triclosan released from the filtering layer. The filter media can be used in various applications including filter closures for facemasks.

### CROSS-REFERENCE TO RELATED APPLICATIONS

This applications claims the benefit of U.S. provisional application 61/072,915, filed on Apr. 3, 2008.

### BACKGROUND OF THE INVENTION

The majority of facemasks and surgical facelets presently on the market consist of fluid resistant material to protect the user from the exposure to aerosols/moisture in the surrounding atmosphere, but do not provide any antimicrobial protection. Recently, efforts have been made to develop antibacterial facelets and facemasks by incorporating an active antibacterial agent in the filtering material (“substrate”) used to manufacture the facelet or facemask. The substrate is usually a nonwoven material consisting of fibers that are bonded together to form a three-dimensional web. The antimicrobial agent may be embedded within the three-dimensional matrix of the substrate or within the fiber itself. Alternatively, the antimicrobial agent may be chemically bonded to the substrate.

One broad-spectrum antibacterial agent known as triclosan (2,4,4′-Trichloro-2′-hydroxydiphenyl ether) has been used as an active agent in antibacterial facemasks. For example, U.S. Pat. No. 7,044,993 (“the '993 patent”) and U.S. Publication No. 20030205137 (“the '137 publication”) describe the manufacture of masks equipped with a network of polyvinyl chloride (PVC) based organic fibers containing triclosan. In the '137 publication, the triclosan is impregnated within the three-dimensional matrix of the network of PVC fibers. WO 2006/034227 (“the '227 PCT”) describes the manufacture of a medical facemask comprising a central transparent portion and an outer filter portion, wherein triclosan is incorporated into one or more regions of the mask. The entire contents of the '993 patent, the '137 publication and the '227 PCT are hereby incorporated by reference.

Triclosan has several important advantages that are useful in filtering applications, including facemasks. Triclosan has a broad range of activity that encompass many types of gram-positive and gram-negative non-sporulating bacteria and some fungi. Moreover, triclosan is highly efficacious at low concentrations so only a small amount is needed for powerful antibacterial activity. In certain settings, including hospitals, triclosan has been shown to be effective in preventing infections.

Despite the advantages of using triclosan as an antimicrobial agent, there are several disadvantages that limit its utility. Some of the problems associated with triclosan include:

It can get into wastewaters that can form into dioxins (an environmental pollutant)

It can form chloroform gas in the presence of chlorine

Its intermediates can be converted into highly toxic dioxins

It is a potential endocrine disruptor

It has the potential to cause bacterial resistance

The Environmental Protection Agency (EPA) has designated triclosan as a polychlorinated biphenyl (PCB) pesticide (PC code 054901). The test method that can be used to quantify triclosan is based on the EPA Compendium Method TO-10A, Determination Of Pesticides And Polychlorinated Biphenyls In Ambient Air Using Low Volume Polyurethane Foam (PUF) Sampling Followed By Gas Chromatographic/Multi-Detector Detection (GC/MD).

Owing to the potential environmental and safety concerns associated with triclosan, using triclosan in connection with various filtering applications is not currently feasible. For example, we have found that antimicrobial facemasks described in the prior art, such as those described in the '137 application, do not effectively limit the release of triclosan into the environment. Thus, an unacceptable quantity of triclosan is liberated from the facemask, exposing the user to a potentially dangerous amount of triclosan and its chemically degraded byproducts.

Given the shortcomings of the prior art, it would be advantageous to develop an antimicrobial filter using triclosan as an active agent, which can be used in various filtering applications (e.g. facemasks). The triclosan-containing filters must be highly efficacious, environmentally friendly and nontoxic. The present invention comprises nontoxic, environmentally friendly triclosan-containing antimicrobial filters composed of two layers, a filtering layer and a backing layer used to trap any triclosan released from the filtering layer.

### BRIEF DESCRIPTION OF DRAWINGS

FIG. 1 is a schematic view of a facelet, in accordance with one embodiment of the present invention.

### DETAILED DESCRIPTION OF THE INVENTION

The following sections describe exemplary embodiments of the present invention. It should be apparent to those skilled in the art that the described embodiments of the present invention provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present invention as defined herein and equivalents thereto.

The present invention is directed to a nontoxic, environmentally friendly antimicrobial filter that uses triclosan as an active agent. The filter comprises at least two distinct layers, one layer containing a substrate (“the substrate”) with embedded triclosan, and a second layer (“backing layer”) which is used to trap any triclosan released from the first layer. Accordingly, the backing layer prevents any triclosan from being released into the environment. As such, a sufficient concentration of triclosan can be used to deactivate/kill bacterial without the concurrent release of toxic chemicals into the environment.

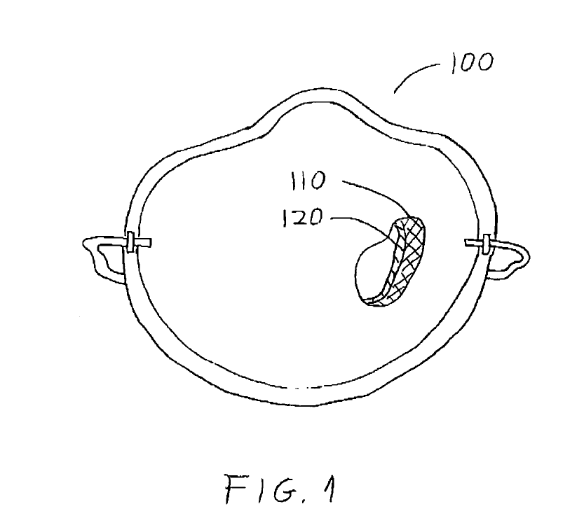

FIG. 1 shows an exemplary embodiment of a surgical facelet of the present invention. The facelet 100 is comprised of two layers, which are depicted in the cut-away view. The front layer 110 is comprised of a nonwoven or other material which contains a sufficient amount of triclosan to exert a toxic effect on a wide-array of bacteria. Backing layer 120 traps any triclosan that is released from front layer 110. Thus, the triclosan is not released into the environment.

The substrate containing the triclosan may be made from a variety of different materials. In a particular embodiment, the substrate may be a fiber-based material having a fibrous matrix structure; it may be a sponge like material having an open cell matrix structure; it may be flexible or inflexible; etc.

In one preferred embodiment, the substrate is a nonwoven fabric. Nonwoven is a type of fabric that is bonded together rather than being spun and woven into a cloth. It may be a manufactured sheet, mat, web or batt of directionally or randomly oriented fibers bonded by friction or adhesion; it may take the form of a type of fabric.

In another embodiment, the substrate may be a nonwoven textile of varying fluffiness, comprising polymer fiber. The polymer may be for example, nylon, polyethylene, PVC, polystyrene polypropylene, polyester, etc. or any other polymer suitable for a filter substrate. Additionally, the substrate can be made of materials other than polymer fiber.

The nonwoven material may be of a type suitable for a high efficiency particulate air filter (i.e. a HEPA filler). A suitable nonwoven material may be obtained from Technol Aix en Provence Cedex 03 France (see Canadian patent no. 1,243,801); another suitable material may also be obtained from Minnesota Mining & Manufacturing Co. (3M). The nonwoven material has a three-dimensional structure which should be configured in such a fashion as to provide a matrix capable of entrapping (i. e. physically) the desired active agent. For example if the nonwoven material is based on fibers, the structural fibers of the nonwoven material may be present and distributed in such a fashion as to provide a fibrous matrix structure able to entrap the desired active agent. The nonwoven material may have a microstructure. In a particular embodiment, the active agent has a size appropriate to be entrapped by the three-dimensional (e.g. web) matrix structure of the desired nonwoven material.

Alternative substrates may further include glass fibers and fibers, such as cellulose, that are ultimately formed into a paper-based filter media. The present invention is not limited to a nonwoven material. Other suitable substrates may include spongy materials or foam.

Additionally, the substrate may be a porous dielectric carrier. An electrostatic charge may be applied across the entire dielectric carrier or a part of the dielectric carrier. Methods of incorporating an electric charge into a dielectric carrier are described in U.S. Publication No. 2006/0251879 (the '879 publication), to the inventor The entire contents of the '879 publication are incorporated herein by reference.

Various methods of incorporating triclosan into the substrate may be used. Several of these methods are described in the '879 publication. For example, the triclosan can be physically entrapped or embedded within the three-dimensional matrix of a nonwoven web. Alternatively, the triclosan may be embedded within the fibers of the nonwoven web.

The backing layer may be composed of the same material as the substrate containing the triclosan. Alternatively, the backing material may be composed of a different material as the substrate containing the triclosan. In a particularly preferred embodiment, the backing layer is a nonwoven. Suitable nonwoven materials to use include but are not limited to polycottons, polypropylene, and polyethylene materials. Properties of the materials can vary in concentration, thickness, pressure drop, and electrostatic charge.

The backing layer contains a powdered additive of varying micron size which is used to trap (scavenge) any triclosan released from the substrate. The additive may be any material, particularly a sorbent, which is capable of scavenging the triclosan by means of absorption or adsorption. A sorbent is defined as a material that sorbs another substance; i.e. that has the capacity or tendency to take it up by either absorption or adsorption. Types of sorbents that can be considered to act as additives may include (but not limited to): molecular sieves, activated carbon materials (coconut and/or coal based), polymers, or a combination of each. A molecular sieve is a naturally occurring or synthetic zeolite characterized by the ability to undergo dehydration with little or no change in crystal structure, thereby offering a very high surface area for adsorption of foreign molecules. A zeolite is any one of a family of hydrous aluminum silicate minerals, whose molecules enclose cations of sodium, potassium, calcium, strontium, or barium, or a corresponding synthetic compound, used chiefly as molecular filters and ion-exchange agents. Activated carbon is a highly absorbent carbon obtained by heating granulated charcoal to exhaust contained gases, resulting in a highly porous form with a very large surface area. It is used primarily for purifying gases by adsorption, solvent recovery, or deodorization and as an antidote to certain poisons.

A common functionality of the additive is to “trap” and retain the triclosan molecule from the airstream. Molecular sieves that can be considered as candidates for use must have a pore size large enough to be able to hold the triclosan molecule, such as Type 13X which has a pore size of approximately 10 Å. Activated carbon must have the capability to collecting triclosan on the surface of its pores such as CBX (coal) and Anasorb 747 (coal and coconut). Additive polymers (either porous or not) used need to have attractive interactions (ranging from weak to strong) between the triclosan species to the surface material. Examples of polymer sorbents include: Tenax TA, Poropak sorbents, and HayeSep sorbents (porous polymers) Anasorb 727 (polystyrene-based polymer), and XAD 2 (polystyrene/divinyl benzene-based polymer).

Other categories of sorbents that can also be considered for use are: charcoal, graphitized carbon, carbonized molecular sieves, silica gels, and other resins. The backing additive is not limited to the use of only one type of sorbent; a combination of sorbents can be use to enhance scavenging capabilities.

The additive applied to the backing layer is preferably in powder form. Manufacturing the backing layer containing additive may be completed by means of coating the material. Coating the material may be performed by applying the additive powder (via dusting or spraying) on the topside of the backing layer. At the same time, a low vacuum is applied to the underside of the backing material in order to “pull and trap” the particles deep within the layer to prevent any loss of additive powder.

After generating the substrate with embedded triclosan and the backing layer with the scavenging agent, the two layers must be combined into a laminate. The two layers may be combined by any suitable means. For example, the two layers may be glued together. Alternatively, the two layers may be combined by ultrasonic welding.

When the backing layer containing the triclosan scavenger is applied to the substrate/triclosan layer, the amount of triclosan released into the air is minimized (see Examples below). Without the backing layer, a significant amount of triclosan is released into the air. Hence, the laminates described herein can be effectively used as antimicrobial filters without the disadvantage of toxic chemicals being released into the environment. The toxic vapors are effectively trapped by the backing layer and thus, will not be inhaled by a person in the vicinity of the filter.

In one preferred embodiment, the triclosan-containing filters of the present invention can be used as facemasks or surgical facelets without risk to the user. Alternatively, the triclosan-containing filters may be part of a facemask, such as the facemask described in the '993 patent and '137 publication. Unlike the facemasks described in the '993 patent and '137 application, using the filters of the present invention minimizes the amount of toxic chemicals released into the environment.

In another preferred embodiment, the triclosan-containing filters of the present invention may be used in an air duct to filter air passing through. For example, the filters may be adapted to align the walls of an air duct and thus deactivate any microbes in the air that encounter the triclosan. The filters may be applied to ventilation systems in houses, hospitals or office buildings. The presence of the backing layer effectively prevents any triclosan from leeching into the environment.

In another embodiment, the filter media according to the present invention can be used as a closure or to make a filter closure for air filters for products such as facemasks and HVAC.

### EXAMPLES

We compared the inventive facelets with a facelet that is currently available in the market. Noveko 3xEZ facelets (“Noveko facelets”) contain triclosan, which is used to control airborne infections and microbes. It is produced by Noveko International Inc. and distributed by Médi-Sélect Ltée. We determined the amount of triclosan applied on the material, the triclosan air concentration released from the use of the facelets, nor the health impact in regards to triclosan exposure (see below).

To evaluate the potential exposure to triclosan using the Noveko facelets, air testing was performed to demonstrate the triclosan concentration released to a potential individual. Air testing parameters are designed to mimic a passive breathing rate of 85 Liters per minute (LPM) in environmental condition of 25° C. and 60% humidity. In addition to testing the Noveko facelets, the innovator facelets with the added backings were evaluated to demonstrate their effectiveness in removing triclosan from the air stream and minimizing any chemical exposure.

This quantitative test method is designed to measure the pesticide and polychlorinated biphenyls (PCBs) found in ambient air using a low volume sampling procedure based on the absorption of chemicals on a combination of polyurethane foam (PUF) and granular sorbent (tenax) (purchased from SKC Inc.). This quantitative test method is designed to measure the pesticide and polychlorinated biphenyls (PCBs) found in ambient air using a low volume sampling procedure based on the absorption of chemicals on a combination of polyurethane foam (PUF) and granular sorbent (tenax) (purchased from SKC Inc.). Based on the specifications of the sample collector tubes (PUF/tenax/PUF sample tubes), a collection rate can range from 1 LPM to 5 LPM for a period of 4 hours to 24 hours. Therefore, the collected air volume per tube can range from 240 L to 1500 L.

For in-house testing of the Noveko facelet with and without additive backing, the test flow through the filter samples (effective surface area of 100 cm2) was at 85LPM using environmental conditions of 25° C. and 60% humidity to mimic passive breathing conditions. PUF/tenax/PUF sample tubes were placed on the effluent air stream (i.e. air flow after the test filter) to collect air samples at a rate of 1LPM for a test period of 7 hours.

Samples were analyzed as per the test method EPA Compendium Method TO-10A. The limit of detection for analysis was 0.2 ug of triclosan.

At environmental conditions (25° C. and 60% humidity) and at a flow rate of 85LPM, a total of 6 facelets were tested to evaluate the amount triclosan that can be liberated during regular use. In three other samples, an additional backing of a triboelectric nonwoven layer, HP50, containing a 100 g/m2 powdered additive (tenax TA absorbent, Type 13X MOLSIV, or CBX Lantor carbon) was applied to the facelet to determine if they can trap the triclosan and prevent its release. Triboelectric is defined as an electric charge produced by friction between two objects.

In addition to air testing of the Noveko facelets, a 100 cm2 facelet sample was cut and analyzed to quantify the amount of triclosan that was applied to the material. This is no indication of what is released in the air stream. An amount found on the material sample will indicate how much triclosan was applied on that given area. Based on this information, one can correlate the amount (μg) of triclosan found on the material to the triclosan released in the air stream (i.e. air concentration; μg/m3).

Samples were extracted and analyzed by GC/MS (as per EPA TO-10A) and the amount of triclosan was determined in μg. Based on the air volume collected during testing of 420 L (with the exemption of the material sample analyzed), the triclosan air concentration (μg/m3) was determined. Full results and a summarized format (which averages results of the six Noveko facelets) are presented in Table 1 and Table 2, respectively.

TABLE 1
Amount of Triclosan liberated from 100 cm2 facelets
(with and without added backing) at a flow rate
of 85 LPM at set environmental conditions.

| | | Triclosan |
|---|---|---|
| | Measured | air |
| | Triclosan | concentration |
| Sample | (μg) | (μg/m³) |
| Noveko Facelet #1 | 2 | 4.8 |
| Noveko Facelet #2 | 2.5 | 6.0 |
| Noveko Facelet #3 | BDL | BDL* |
| Noveko Facelet #4 | 2.1 | 5.0 |
| Noveko Facelet #5 | 2.4 | 5.7 |
| Noveko Facelet #6 | 0.94 | 2.2 |
| Facelet & 100 g/m² Tenax 35/60 | BDL | BDL* |
| mesh HP50 | | |
| Facelet & 100 g/m² Type 13X | BDL | BDL* |
| MOLSIV HP50 | | |
| Facelet & 100 g/m² CBX Lantor | BDL | BDL* |
| Carbon HP50 | | |

BDL = Below the detection limit of 0.2 μg of triclosan
BDL* = BDL calculated air concentration of 0.5 μg/m³

TABLE 2
Summary of Air Results in the Amount of Triclosan liberated
from 100 cm² facelets (with and without added backing) at
a flow rate of 85 LPM at set environmental conditions.

| | Triclosan air |
|---|---|
| | concentration |
| Sample | (μg/m³) |
| Facelet (n = 6) average concentration | 4.7 |
| Facelet & 100 g/m² Tenax 35/60 mesh HP50 | BDL* |
| Facelet & 100 g/m² Type 13X MOLSIV HP50 | BDL* |
| Facelet & 100 g/m² CBX Lantor Carbon HP50 | BDL* |

BDL* = BDL calculated air concentration of 0.5 μg/m³

Based on the results of the 100 cm² samples, it was determined that the Noveko facelet contains an actual measured amount of 1100 μg of triclosan found only on the material. Of the present 1100 μg triclosan found on the sample material, an average air concentration of 4.7 μg/m³ of triclosan was released upon exposure. But when an additive backing layer was added to triclosan facelet material, the triclosan exposure concentration decreased to below detection limits (BDL) of 0.5 μg/m³.

To evaluate the full exposure of triclosan from a Noveko facelet, one can extrapolate the preliminary results to the fill-size facelet On a reduced area of 100 cm², 1100 μg of triclosan is measured with an average air concentration of 4.7 μg/m³. Extrapolated results for a full facelet with an effective area of 248 cm² yields an approximately 2728 μg of triclosan with an average air concentration exposure of 11.7 μg/m³.

At present, there is a lack of documentation regarding the acceptable exposure limit of triclosan and any health risks it imposes. Therefore, assessment of the exposure concentrations may be premature. But with the knowledge of adding an additive backing layer, one can conclude that this is a novel method to reduce/eliminate triclosan exposure for an individual user.

What claimed is:

1.) A surgical facelet, comprising:
a front layer impregnated with a quantity of triclosan for disinfecting a stream of air passing through said facelet; and
an air permeable backing layer disposed to receive air passing through said front layer, said backing layer containing a sorbent capable of scavenging any triclosan released from said front layer.
